(19) United States
(12) Patent Application Publication
(10) Pub. No.: US 2017/0295735 A1
(43) Pub. Date: Oct. 19, 2017

(54) Improved Methods Of Plant Breeding Using High-Throughput Seed Sorting

(71) Applicant: MONSANTO TECHNOLOGY LLC, St. Louis, MO (US)

(72) Inventors: David V. Butruille, St. Louis, MO (US); Roger A. Weyhrich, St. Louis, MO (US)

(21) Appl. No.: 15/511,934

(22) PCT Filed: Sep. 10, 2015
(86) PCT No.: PCT/US15/49344

§ 371 Date: Mar. 16, 2017

Related U.S. Application Data
(60) Provisional application No. 62/051,000, filed on Sep. 16, 2014.

(51) Int. Cl. A01H 1/04; A01H 1/08

(52) U.S. Cl. CPC A01H 1/04; A01H 1/08

(57) ABSTRACT

The present disclosure describes improved methods of plant breeding using rapid seed sorting processes. More particularly, it relates to the application of automated systems and methods that rapidly and nondestructively measure, classify, and sort seeds to improve the rate, efficiency, and accuracy of selection decisions and other processes related to improving crop genetics.

### CROSS-REFERENCE TO RELATED APPLICATIONS

This application is the US national stage under 35 U.S.C. §371 of International Application No. PCT/US2015/049344, filed on Sep. 10, 2015, which claims the benefit of U.S. Provisional Application No. 62/051,000, filed on Sep. 16, 2014. The entire disclosure of the above applications are incorporated herein by reference.

### FIELD

The present disclosure relates generally to the use of high-throughput seed sorting systems in a plant breeding program. More particularly, it relates to the application of automated systems and methods that rapidly and nondestructively measure, classify, and sort seeds to improve the rate, efficiency, and accuracy of selection decisions and other processes related to improving crop genetics.

### BACKGROUND

Haploid seed sorting is a process wherein a population of seeds potentially containing haploid embryos (“haploid seeds”) are identified and separated from seeds that contain embryos of a higher ploidy level (e.g. diploid), to create a new population of seeds that are substantially all haploid. This process is necessary in a large doubled-haploid breeding program using in vivo maternal haploid induction to prevent resources from being wasted on seeds containing diploid embryos (“diploid seeds”) that result from induction crosses.

For example, one important step in a doubled-haploid pipeline that requires considerable resources is the process of “doubling” the ploidy of a plant, i.e. doubling the number of chromosomes in at least one cell of a haploid plant. Plant breeders currently pursuing efficient doubled-haploid pipelines seek to allocate resources, (e.g. the resources required for chromosome doubling) to only haploid seeds that contain desired traits and genetics. Consequently, before breeders subject populations of putatively haploid plants to what are typically expensive, laborious, and often inefficient downstream processes like chromosome doubling, they “haploid sort” the populations they generate from induction crosses in order to identify and discard most diploid seeds. After haploid sorting, the populations are subjected to selection, during which the breeder analyzes the populations to determine which contain certain traits at frequencies the breeder desires to increase or maintain in the breeding program. Thus, the steps of haploid sorting and selection are typically performed before doubling to ensure downstream resources, like those used for chromosome doubling, are allocated only on plants that are most likely to contain the traits desired in the breeding program.

Plant breeders have wrestled with the challenge of scaling up haploid seed sorting for decades, and as a consequence, modern breeding programs are structured to counter the limitations imposed by relatively slow, inefficient, and often inaccurate haploid seed sorting methods presently known in the art. When new rapid haploid seed sorting methods become available, drastic innovations that were previously counter-intuitive will become conceivable, and certain novel rearrangements of the steps breeders must currently take to generate large numbers of doubled haploids will become feasible.

The advantages of using doubled haploids (DH) to create homozygous inbred lines as opposed to using conventional crossing methods, such as recurrent selfing or sib-mating, are well known (Nei 1963; Melchinger et al. 2005, 2013; Geiger et al. 2009). In vivo maternal haploid induction is commonly used to generate DH plants and involves pollinating a donor plant containing desired genetics by an “inducer” that, at some frequency, induces the formation of seed with haploid embryos (i.e. haploid seeds). However, attempts to scale up DH production using this method are hampered by the fact that typically most of the seed produced in an induction cross is not haploid. Thus, very large numbers of seeds must be sorted in a commercial DH pipeline to avoid wasting vast resources and time on unwanted seeds at later steps in the pipeline such as the chromosome doubling step.

This problem is compounded by the fact that the outward differences between seeds of differing ploidy levels are naturally difficult or impossible to distinguish with the naked eye for most species. In corn, for example, haploid and diploid seeds are virtually identical in outward appearance, so a considerable portion of the total resources allocated to DH production are currently spent on the single step of haploid seed sorting.

One standard haploid sorting approach is to hire teams of human sorters to manually identify the presence or absence of visible genetic color markers carried by the male inducer that manifest in the tissues of any seed the inducer fertilizes. The dominant R1-nj marker is often used for this purpose. Other published methods attempt to mitigate the problem by using transgenic marker systems to more clearly mark haploid seeds as set forth, for example, in US 2008/0189801. However, these methods do not solve the haploid seed sorting bottleneck as they are also based on sorting seeds at relatively low speeds. Furthermore, the use of transgenic markers may lead industrial breeding pipelines to face additional regulatory certifications before the commercialization of product derived through this approach. For these and other reasons, large DH pipelines remain focused on mitigating the limitations imposed by haploid seed sorting bottlenecks.

Despite repeated attempts to eliminate the bottleneck, it has been a central issue of DH technology for decades, and breeding programs are routinely structured around the problem. For example, a central task of every large DH program is selecting the haploids which will be planted and evaluated in the next cycle. Because haploid seed sorting consumes so much time and resources, standard methods in the art teach that haploid seed sorting in large programs should be performed only after researchers have determined which pools of haploid progeny contain the traits and/or genetics they desire in the breeding program, i.e. that selections among populations must be made before seed sorting. This order of operations is necessary to avoid wasting time and resources sorting seeds that will be eliminated later on and is a requirement to running a large and efficient DH pipeline.

Another critical issue for large DH pipelines is ensuring that the haploid seeds selected for the next generation are prepared ahead of planting deadlines. Planting deadlines are often set by immutable environmental limitations of climate, weather, or set by the expected performance of the seeds being planted. Current large breeding pipelines must be structured to accommodate the long process of haploid seed sorting, and so the haploid sorting step must be initiated several weeks ahead of any planting deadlines to ensure that haploid seed populations are ready at the optimal planting time. This prevents breeders from performing more extensive analyses that would otherwise improve selection accuracy, which further increases the likelihood that downstream (e.g. doubling) resources will be wasted on plants that lack at least one selection criterion set by the breeder. As a result, breeders using current haploid sorting methods commonly find themselves forced to rely on subjective criteria when making decisions in order to meet planting deadlines. This is because they must start the relatively slow process of sorting before they have completed more objective analyses based on, for example, genotyping, sequencing, or statistical assays. Such subjective selection reduces the long-term success and efficiency of large breeding programs but is nevertheless currently considered a necessary aspect of creating large numbers of DH.

New advancements in automation and rapid seed-sorting and analysis systems provide innovative opportunities to redesign today's plant breeding programs to efficiently incorporate the advantages of these new technologies. As described herein, the term “rapid” as used to describe a seed sorter, especially a seed sorter capable of identifying and/or distinguishing haploid seeds from other types of seed (i.e. a haploid seed sorter), can be used synonymously with “high-speed” and “high-throughput”, and is meant to distinguish seed sorting at rates that are substantially faster (e.g. more than 5 seeds per second) than manual methods.

The advantages of these new approaches were not previously considered because rapid haploid seed sorters capable of sorting several seeds per second, like that described in US 2014/0266196 or other systems that detect signals alternative to NMR, were not available such that breeding programs without an obligate bottleneck at the seed sorting step could not be realized. Moreover, although the advantages of the methods disclosed herein are widely applicable, only those implementing large DH pipelines requiring the sorting of hundreds, thousands, tens of thousands, or more putatively haploid populations in a given breeding cycle would be motivated to expend resources researching in this area.

Innovation and restructuring of large breeding programs are not economical in without-rapid sorting, but become much more viable with rapid seed sorting. Thus, rapid seed sorting allows the restructuring of traditional breeding programs and permits the breeder to alter the order of operations and timing of breeding cycle steps to improve selection accuracy and maximize DH production potential. Accordingly, innovative methods of incorporating rapid seed sorting systems into breeding programs in order to satisfy those objectives are disclosed herein.

### SUMMARY

Provided herein are various methods of integrating rapid seed analysis and seed sorting systems into a plant breeding pipeline. More particularly, it discloses how automated systems and methods that rapidly and nondestructively measure, classify, and sort seeds can be incorporated into a DH pipeline, along with novel improvements to current breeding practices what maximize the benefits of rapid seed sorting.

Methods disclosed herein include improving the efficiency of a breeding pipeline comprising creating a first set of at least two distinct haploid plant populations in the form of seeds, such that at least one population of the set comprises a frequency of haploid embryos; sorting at least two plant populations in the first set using a high-speed sorter to create a second set of distinct populations, each distinct population in the second set comprising a frequency of haploid embryos that is greater than the frequency of haploid embryos in the corresponding set 1 population from which the second population was derived; determining the likelihood that a plant appearing in set 1 contains at least one trait; and selecting at least one plant from at least one population in set 1 to advance in a breeding pipeline based on the outcome of step c.

In certain embodiments, sorting the populations of haploid seeds in set 1 is performed during or prior to selecting which populations will be advanced in the breeding pipeline. In certain embodiments, the sorting step is performed prior to determining the likelihood that a plant appearing in the set 1 contains at least one trait. In certain embodiments, at least one of the haploid plant populations in set 1 is generated by maternal haploid induction. In certain embodiments, the advancement of at least one population in the breeding pipeline comprises doubling the chromosome number of at least one cell of at least one embryo that appeared in set 1. In certain embodiments, the step of determining which plants contain a desired trait is performed on plants after the plants have germinated. In certain embodiments, the step of determining which plants contain a desired trait comprise assessing the yield potential, disease resistance, or other performance data of a parent, ancestor, progeny, or other related germplasm growing in a field, greenhouse, or other area suitable for growing plants. In certain embodiments, genotyping is used to determine whether a plant contains a certain trait. Such genotyping can employ the use of a molecular marker to signal the presence/absence of a certain trait. This can also involve detecting or quantifying the presence of a nucleic acid sequence produced by a plant in set 1. In certain embodiments, an NMR analysis is used to haploid sort seeds, and in certain embodiments the NMR analysis can be used to determine whether a seed contains a certain oil content. In certain embodiments, methods described herein are performed on “large” breeding programs wherein more than 10,000 seeds are haploid sorted in any 12-month period.

Certain embodiments described herein include methods of creating populations of doubled haploid plants in a doubled haploid production pipeline wherein a rapid haploid seed sorter is used to sort populations of haploid seeds and wherein the number of haploid populations sorted is greater than the number of haploid populations that are advanced in the breeding pipeline. In certain embodiments, advancing a population in the breeding pipeline comprises doubling the chromosome number of at least one cell of at least one embryo in the population. In certain embodiments, the number of induced haploid populations exceeds the number of populations doubled in the breeding pipeline by 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, or more than 250%. In certain embodiments, the at least one population of haploid seeds sorted by a rapid seed sorter is derived from a maternal haploid induction cross.

Other embodiments described herein provide high throughput methods for bulking a population of doubled haploid seeds wherein a first population of seeds comprising haploid seeds is sorted using a rapid haploid sorter system to accumulate a second population of seeds comprising haploid seeds that is substantially devoid of diploid seeds. The second, accumulated population of haploid seeds can be analyzed, either phenotypically or genotypically, and one or more individual seeds exhibiting at least one preferred characteristic from the second population of seeds can be selected. Doubled haploid seeds are then produced from the selected seeds; and plants or plant tissue is cultivated from the selected doubled haploid seeds

The foregoing and other objects and aspects of the invention are explained in further detail in the specification and examples below.

### DETAILED DESCRIPTION

The following description is merely exemplary in nature and is in no way intended to limit the present teachings, application, or uses. Various modifications to the disclosed embodiments will be readily apparent to those skilled in the art, and the general principles defined herein may be applied to other embodiments and applications without departing from the spirit and scope described herein. Thus, the present invention is not intended to be limited to the embodiments shown, but is to be accorded the widest scope consistent with the principles and features disclosed herein.

Embodiments described herein include methods of using rapid seed-sorting devices to accumulate pools (or populations) of haploid seeds substantially devoid of diploid seeds. Particular embodiments of rapid seed sorting devices suitable for use with the methods of the present invention are described in co-owned U.S. patent application Ser. No. 14/206,238 (filed Mar. 12, 2014), which is incorporated herein by reference in its entirety. For example, in some embodiments of the present methods, a rapid seed-sorting device using an NMR signal is employed to rapidly distinguish and sort haploid vs. diploid seeds, for example, as described in U.S. patent application Ser. No. 14/206,238. In other embodiments, other suitable devices for the rapid sorting of seed can be used.

Following rapid haploid sorting, selected seeds can be doubled using any chromosome doubling method known in the art, including high-throughput and/or automated doubling systems and methods.

A rapid haploid seed sorter could be used at any point in a breeding program where a user desires to separate seeds based on their ploidy level. Applicants do not intend to limit the use of rapid haploid seed sorting systems for sorting populations of seeds only derived from induction crosses.

In one embodiment, the systems and methods described herein allow for new methods of “mass sorting” haploid seed, described herein as using a rapid haploid sorter to sort populations of seeds before or during the time that selection decisions among the populations are made, i.e. before or while the process of determining which populations contain at least one seed with at least one characteristic at a frequency the user desires to maintain, increase, or decrease in a breeding program are performed. In one embodiment, a breeding program employs a rapid seed sorter to sort induced haploid populations as part of a “DH pipeline”, a multi-step process that doubles the chromosome numbers of at least one cell of a haploid plant. These analyses could include any evaluation, test or ranking system that influences selection decisions, including characterizing the induction rate (i.e. induction “performance”) of an induced hybrid, or data characterizing the breeding value of one or more parents of an induced hybrid (e.g. an inbred's general combining ability), or chipping and genotyping at least one seed of at least one induced haploid population. In contrast to current methods known in the art, a user employing mass sorting will typically expend a large portion of their total haploid sorting resources on populations that the user realizes will later fail one or more selection thresholds set by the user, and thus will not be selected for advancement to the next breeding cycle. Mostly due to costs, this approach is counter-intuitive using current methods in the art, but rapid haploid seed sorting systems and devices will make mass sorting a practical and worthwhile strategy in a large breeding program.

In certain embodiments, the term “large”, when used to describe a breeding program, breeding pipeline, and/or a DH pipeline, can include any series of steps made by an institution, corporation, machine, system, or group of at least one person wherein more than 10,000 seeds are subjected to haploid sorting techniques or technologies during any 12-month period. In certain embodiments, a large breeding pipeline is one that haploid sorts more than 10,000, 20,000, 30,000, 40,000, 50,000, 60,000, 70,000, 80,000, 90,000, 100,000, 110,000, 120,000, 130,000, 140,000, 150,000, 160,000, 170,000, 180,000, 190,000, 200,000, 210,000, 220,000, 230,000, 240,000, 250,000, or more than 250,000 seeds in any single 12-month period.

In certain embodiments, the systems and methods described herein allow for the “late sorting” of seed, wherein rapid haploid sorting is implemented after or during the time when selections among potential haploid populations are made and allows for time to collect and analyze additional phenotypic, genotypic and/or other information. Incorporating a rapid haploid sorter after selection permits a user to make a large number of accurate selections based on analyses completed after harvest and before planting deadlines. This is because a user can sort so rapidly, e.g. 5 or more seeds per second, that only minutes or hours are typically needed to sort an entire induced haploid population comprising thousands, tens of thousands, or more. This moves the deadline to start sorting considerably closer to the planting deadline, making available significant savings in time that can be re-allocated to other processes, such as improving selection accuracy. Late sorting has the added advantage of saving sorting resources until after it is known which populations will satisfy the selection criteria set by the user.

In still a further embodiment, the systems and methods described herein enable “over induction”, defined as a breeding strategy wherein significantly more induction progeny seed is generated in a DH pipeline than are subjected to the subsequent doubling steps of the pipeline. Thus, over induction effectively relaxes the selection intensity set by limitations of doubling and other downstream DH-related processes. This is because incorporating a rapid haploid seed sorter into a breeding pipeline frees up considerable time that can be reallocated to performing highly accurate selections between the time the induction populations are planted, harvested, and processed and planting deadlines. As a consequence, more false positives will be eliminated (as defined here, a false positive is a population that is selected, but in reality fails to meet a selection threshold set by the user), which in turn frees up more resources that can be applied to performing more induction crosses and testing a greater number of haploid progeny. This additional objective testing, in turn, results in fewer false negatives that would have been eliminated if manual sorting methods were used (as defined here, a false negative is a population or seed that is not selected, but in reality satisfies the selection threshold set by the user). Thus, over induction is based on the realization that a rapid haploid seed sorter can actually make it more efficient to sort large numbers of seeds that are unlikely to be selected for advancement at later points in the breeding pipeline (e.g. for example, planting and/or reproduced to increase in number, selfing or crossing with other plants, chromosome doubling, and/or transformation). This idea is new because rapid haploid sorting now makes it possible to perform more accurate selections utilizing new information resulting from performance tests of the parents, ancestors, or progeny of the populations in the breeding pipeline (e.g. field yield testing) and still meet target planting deadlines and/or meet deadlines to perform other steps later in the pipeline (e.g. doubling).

Selection methods and devices for determining whether seeds contain a desired trait could be used in conjunction with rapid haploid seed sorting, such as those that collect image data, (e.g. U.S. Pat. No. 8,253,054, U.S. Pat. No. 7,600,642, and/or a color seed sorter (e.g. Color Seed Sorter by National Manufacturing), and/or systems and devices that that chip and/or genotype seed (e.g. U.S. Pat. Nos. 7,611,842, 7,830,516, and 7,685,768), and/or an automated seed counter (e.g. the elmor 650 Multi-Channel Counter), or any other device that can be used to detect or identify seeds or evaluate plant phenotypes, genotypes, or performance.

In other embodiments, a rapid haploid seed sorter could be used to assess the performance of an inducer. For example, a rapid haploid seed sorter that assesses the oil content of seeds derived from crosses with a high-oil inducer, e.g. as described in U.S. patent application Ser. No. 14/206,238, could be used to determine whether hybrid seeds derived from a particular inducer cross lack a specified threshold of oil content. In this way, inducers that generate offspring that are difficult to haploid sort could be efficiently eliminated and substituted by better performing inducers.

A rapid haploid seed sorter can also be used to assess inducer performance based on induction rate. For example, a rapid seed sorter could be used to rapidly determine the induction rates derived from inducing a set of hybrids with a particular inducer. This would rapidly provide accurate induction rate performance data for a given inducer and permit a user to rank an inducer's performance relative to the induction rates of other inducers tested in a similar way. These types of tests can also be used to evaluate the ability of a given population to be induced. Test results ranking a given population's tendency to perform better with a given inducer, or among a set of inducers, relative to other materials can be used to drive future selection decisions about the parents of that population and other materials tested in a similar way.

Embodiments of this invention also include using a rapid haploid seed sorter to sort seeds based on any characteristic the sorter can measure, regardless of whether the characteristic is linked to ploidy level. For instance, a population of hybrid seeds derived from crossing two elite inbreds could be screened to identify and select those seeds which exhibit a threshold level of any trait the rapid haploid seed sorter can measure. In one example, the rapid haploid seed sorter described in U.S. patent application Ser. No. 14/206,238 detects an NMR signal and weight for each seed that passes through it. Consequently, such a system could be used to screen any population of seeds to identify and select those exhibiting a threshold value of any characteristic an NMR signal can measure and differentiate, such as weight, water content, or oil content. Other systems that haploid sort using technologies alternative to NMR could be similarly adapted to assay and sort seeds based on any criterion the sorter uses to identify and distinguish.

It also is envisioned that concepts described herein could be used in conjunction with rapid seed sorters that detect ploidy level by quantifying the frequency of nucleotide sequences among a population of seeds, e.g. as described in U.S. patent application Ser. No. 13/819,490, which is incorporated herein by reference in its entirety. Thus, embodiments of this invention are not limited to the specifics of any particular type of rapid haploid seed sorter, and could be applied to sorters that use visible light, NMR, X-ray, MRI, or any other kind of signal statistically correlated to the presence of a trait the user desires to measure. In one embodiment, the high-speed sorter detects the presence of at least one reporter molecule that binds to at least one specified nucleic acid or amino acid sequence that the user wishes to use to differentiate the seeds in a population. In other embodiments, the high-speed sorter is used to differentiate seeds based on the presence or absence of particular isotopes, e.g. C12 vs. C14. In some embodiments, this detection is accomplished by the use of rapid mass spectrometry.

Furthermore, rapid seed sorters could be used to sort and/or phenotype seeds for multiple traits at substantially the same time for any combination of traits the sorter is able to identify and discriminate. For example, it is envisioned that a rapid seed sorter using NMR technology to detect oil levels could sort seeds based on ploidy, and also sort seeds based on their water content as they pass through the device. In this embodiment, a user could set an NMR sorter to rapidly sort a population of seed derived from an induction cross so as to provide the user with a population of pure haploid seed wherein each seed contains no more than a threshold level of water that was set by the user. The data generated by this process could also be used to make other relevant breeding decisions, like parent selection. Rapid haploid seed sorters using technologies other than, or in addition to, NMR could be used to sort seeds based on any criteria the rapid haploid sorter has the capacity to detect and measure.

Additionally, in certain embodiments, a rapid seed sorter could be used in conjunction with any other methods, processes, or technologies useful for sorting or analyzing plants in order to further improve the efficiency of a breeding pipeline. These could be employed in parallel, serial, cyclical, and or substantially any other arrangement that a user desires using various machines in different combinations. For example, a user could employ a rapid optical seed sorter to sort seeds based on a desired characteristic prior to or after the seeds are sorted by a rapid seed sorter using NMR technology. It is also envisioned that multiple different combinations of seed sorting technologies could be employed in the same device, such that seeds are sorted by multiple sections of the same machine wherein each section sorts seeds based on a different set of criteria or technology (e.g. a machine that sorts seeds based on oil properties that are detected by an NMR system of the machine, and sorts seeds based on the size and/or shape of an embryo as characterized by an X-ray imaging system of the same machine).

Embodiments of this invention include mass sorting during or immediately following harvest. In some examples, seeds from induction crosses are sorted within a few hours, or days, of being harvested. In other examples, haploid seed sorting occurs during harvest by a rapid seed sorter deployed in the harvest location. A field-deployed rapid seed sorter sorting seeds based on optical criteria could be mounted directly to a harvester such that seed populations are sorted before leaving the harvester, or before being removed from the cob (shelled). A field-deployed rapid seed sorter unit could also be separable from the harvester and arranged to receive harvested seed from the harvester, e.g. a pull-behind unit or a self-propelled unit. A sorter could also be deployed nearby a field being harvested, such that the seed is shuttled from the harvester in the field to the sorter outside of the field. A rapid seed sorter could also be arranged to receive seed shuttled from multiple fields being harvested substantially simultaneously.

Thus, users will be able to make ready for germination, or planting, populations of haploid seed that satisfy the selection requirements for advancement to the next breeding cycle that are set by the user, all in the field and all within minutes of being harvested. These systems could be configured to transmit data to other locations, like a hand-held device near the harvester or more remote locales like an centralized processing and management center several miles away, such that the entire process could be monitored and controlled remotely. Similarly, it is envisioned that the entire process of harvesting, sorting, genotyping, and selection could be performed without a human operator in the field through the employment of automated vehicles, remote control and/or wireless communication.

This invention is not limited by the number of classes to which a given rapid haploid seed sorter can assign seeds. Rapid haploid seed sorters that can sort seeds into more than two classes are envisioned, which will provide users with more crossing and selection options in subsequent cycles of a breeding program, which can be leveraged to improve the efficiency of the breeding program and increase the rate of genetic gain.

Furthermore, Applicants do not intend that this invention should be limited to any specific crop species. Although some descriptions and examples use corn as a model crop, it is envisioned that concepts disclosed herein could be applied in any situation where a device is used to rapidly sort seed based on possession of a trait, including a trait associated with ploidy level, e.g. the high oil marker used to discriminate haploid vs. diploid seed in U.S. patent application Ser. No. 14/206,238.

As used herein, “germination” describes a point in a plant life cycle that begins when the first root radical emerges from the seed coat and can overlap “sprouting”, during which the seed begins to put out shoots, typically after a period of dormancy.

As used herein, “cultivate” describes any activity that promotes or improves the growth of a plant at any point in its life cycle, including germination.

Further areas of applicability of the present teachings will become apparent from the description provided herein. It should be understood that the description and specific examples are intended for purposes of illustration only and are not intended to limit the scope of the present teachings.

### EXAMPLES

### Example 1

### Mass Sorting Using a Rapid Haploid Seed Sorter

A user of this invention pollinates several thousand corn plants with a high-oil content inducer that reliably produces hybrid progeny seed containing 8% or greater oil content, e.g. UH601, and the putatively haploid seeds resulting from each induction cross are harvested several weeks later. Next, a sample from each population is obtained and then analyzed by a variety of techniques, including automated chipper genotyping (e.g. U.S. Pat. Nos. 7,611,842, 7,830,516, 7,685,768) and automated nucleotide sequencing, and the results analyzed statistically to detect and characterize the presence of potential quantitative trait loci (QTL) and/or informative nucleotide sequences in each population. Before concluding which populations actually meet the user's selection criteria, most or all of the several thousand populations are processed through the rapid haploid seed sorter described in U.S. patent application Ser. No. 14/206,238 to accumulate several thousand populations of pure haploid seeds, most of which do not meet the selection criteria set by the user. These populations of haploid seed that are substantially devoid of diploid seed can be stored using standard methods in the art that preserve seed viability for several days or weeks while the user continues to analyze the populations and determine which are most likely to contain the traits the breeder desires.

Realizing that all induction cross progeny populations are already haploid sorted, a user of this invention will be able to analyze their populations more thoroughly than attempting to process approximately the same number of haploid induction crosses using methods known in the art, which require one to stop analyzing earlier and make selections several weeks sooner in order to sort their selected populations in time for planting. Meanwhile, a user of this invention will have more time to analyze populations with objective tests that improve selection accuracy and reduce the likelihood of selecting false positives and/or eliminating false negatives from their DH pipeline as compared to those using methods currently known in the art. This will lead a user of this invention to experience greater efficiencies and reduce wasting resources on seeds that lack the frequency of desired traits.

### Example 2

### Late Sorting Using a Rapid Haploid Seed Sorter

A user of this invention pollinates several thousand corn plants with a high-oil content inducer that reliably produces hybrid progeny seed containing 8% or greater oil content, e.g. UH601, and the putatively haploid seeds resulting from each induction cross are harvested. Next, a sample from each population is obtained and analyzed with a variety of techniques, including automated chipper genotyping (e.g. U.S. Pat. Nos. 7,611,842, 7,830,516, 7,685,768) and automated nucleotide sequencing, and the results analyzed statistically to detect and characterize the presence of potential quantitative trait loci (QTL) and/or informative nucleotide sequences in each population.

The user of this invention will realize that a rapid haploid seed sorter such as that described in U.S. patent application Ser. No. 14/206,238 can sort approximately thirteen seeds per second, permitting the user to continue running tests and analyzing results that maximize selection accuracy until just a few hours before the planting deadline. Meanwhile, those using current DH production methods will be forced to cut short their tests, make their selections several weeks earlier, and will be unable to subject their populations to the same level of objective selection scrutiny. Thus, a user of this invention will be less likely to select false positives and/or eliminate false negative, leading to greater efficiencies and less resources wasted on seeds that lack the frequency of desired traits as compared to those using methods currently known in the art.

### Example 3

### Over Induction

A user of this invention calculates that their DH pipeline has the capacity to double a maximum 10,000 haploid progeny populations after they are harvested. Using current DH production protocols, the user could count on a selection accuracy of approximately 80%, meaning that doubling resources would be invested into approximately 2,000 populations that will be eliminated when later results reveal that those populations actually do not meet the selection criteria set by the user. On the other hand, by incorporating the methods of this invention, the user will realize that they will be able to free up the time and resources that would have been spent manually sorting seeds and will be able to use that time to improving selection decisions instead. By employing a rapid haploid seed sorter and performing highly-accurate selections among the induced haploid populations before doubling, a user of this invention can process significantly more induction crosses through their pipeline.

It is envisioned that a user in a typical growing season could perform a number of induction crosses that would significantly exceed the number of populations that are actually doubled. In some cases, the number of induced haploid progeny populations generated exceeds the number of populations that are doubled by about 10%, 20%, 30%, 40%, 50%%, 60%, 70%, 80%, 90%, 100%, 110%, 120%, 130%, 140%, 150%, 160%, 170%, 180%, 190%, 200% or any increment in between.

In other examples, the number of induction crosses performed would completely overwhelm the available doubling capacity of current DH pipelines. It is envisioned that a large breeding program would achieve better efficiency by generating a number of induced haploid progeny populations that exceeds the number of populations doubled in the pipeline by a factor of about 3×, 4×, 5×, 6×, 7×, 8×, 9×, 10× or any increment in between.

This enormous potential increase in the number of induced haploid populations that can be evaluated has the potential to revolutionize DH production throughput far above that which would be predicted directly from the increase in sorting speed afforded by a rapid haploid seed sorter. Aspects of this invention make feasible for the first time the ability to harvest, sort, and accurately select huge numbers of seed (e.g. hundreds of thousands, or several million, or more), or huge numbers of induced haploid populations (e.g. several thousand or more) in the same growing season.

### Example 4

### Concurrent Harvest Sorting

A corn harvester is configured to incorporate a rapid NMR haploid seed sorter. In addition to other ways the harvester processes seed, e.g. husking, shelling, etc., seeds are haploid sorted by the on-board rapid seed sorter while the harvester operates in the field. Although it may be necessary for the harvester to stop harvesting and/or traversing the field from time to time, the harvester is typically able to harvest a set number of seeds at approximately the same rate that the rapid seed sorter is able to analyze and sort the same set of seeds, such that there is essentially no point at which harvesting must be slowed or stopped to prevent an accumulation of harvested seeds waiting to be haploid sorted.

A user of this invention employing a machine configured in this way could harvest and sort the induced haploid populations of several thousand or even several hundred thousand, induction crosses growing in a field to obtain a population of pure haploid seed from each population. As the harvester traverses the field, on-board GPS-based computer systems cross reference the location of the harvester with the location of the populations in the field so that systems aboard the harvester can monitor and verify the population from which each seed moving through the harvester was harvested. Processing and packaging systems on board the harvester recombine the haploid seeds within each population to provide a series of containers, each containing a substantially homogeneous population of haploid seeds derived from a single induction cross, and labeled in a way that designates the germplasm of the parents used in that cross.

As soon as a population of haploid seeds has exited the harvester, a sample comprising at least one seed is captured and analyzed to determine whether the sample contains a trait at a frequency the user desires. The user continues to test samples of seeds from at least one of the harvested populations until a few days, or even a single day, or even a few hours before the user begins planting seeds from at least one of the harvested populations, at which point the user concludes their tests and selects the populations of haploid seeds that meet the selection criteria set by the user. Seeds from selected populations are germinated and subjected to doubling using standard methods of the art.

### Example 5

### Concurrent harvesting, sorting, and selection using a rapid haploid seed sorter.

A corn harvester is configured to incorporate a rapid NMR haploid seed sorter that operates on the principles described in U.S. patent application Ser. No. 14/206,238. The harvester is also configured to incorporate a rapid seed chipping and genotyping system based on the principles described in U.S. Pat. No. 7,830,516. The system is configured so that seeds are harvested and processed through the rapid haploid sorter and the chipping and genotyping system all at approximately the same rate, such that there is no backup of seed waiting to be processed at any step.

A user of this invention employing a combine configured in this way could harvest, sort, analyze, and select a population of seeds all substantially concurrently to obtain a population of pure haploid seed from each population, each satisfying both a phenotypic selection criterion set by the user, such as seed weight or water content (e.g. as measured by an on-board NMR sorter) and a genotypic selection criterion (e.g. as determined by the on-board genotyper). As the harvester traverses the field, on-board GPS-based computer systems cross references the location of the harvester with the location of the populations in the field so that systems aboard the harvester can monitor and verify the population and position from which each seed moving through the harvester was harvested. Processing and packaging systems on board the harvester recombine the haploid seeds within each population that satisfy the phenotypic and genotypic criteria set by the user to provide a series of containers, each containing a haploid population of seeds that is substantially devoid of diploid seeds and that has been derived from a single induction cross that satisfies the users selection criteria, and labeled in a way that designates the location in the field from which the seeds were harvested and the parents used in the cross that generated each seed.

Thus, the methods and systems disclosed herein reveal how a user can take full advantage of the improvements that incorporating a rapid haploid seed sorter into a DH pipeline makes available. Application of these concepts, and the alterations necessary to take full advantage of them, require counter-productive alterations in the way large DH pipelines using current or historic haploid sorting methods have been structured and managed for decades, and thus are not currently apparent to those of skill in the art.

The description herein is merely exemplary in nature and variations that do not depart from the gist of that which is described are intended to be within the scope of the teachings. Such variations are not to be regarded as a departure from the spirit and scope of the teachings.

1. An improved method of breeding haploid plants comprising:
a. creating a first set of at least two distinct haploid plant populations in the form of seeds, such that at least one population of the set comprises a frequency of haploid embryos,
b. sorting at least two plant populations in the first set using a high-speed sorter to create a second set of distinct populations, each distinct population in the second set comprising a frequency of haploid embryos that is greater than the frequency of haploid embryos in the corresponding set 1 population from which the second population was derived,
c. determining the likelihood that a plant appearing in set 1 contains at least one trait, and
d. selecting at least one plant from at least one population in set 1 to advance in a breeding pipeline based on the outcome of step c.

2. The method of claim 1, wherein step b. is performed during or prior to performing step d.

3. The method of claim 1, wherein step b. is performed prior to step d.

4. The method of claim 1, wherein at least one of the haploid plant populations in set 1 is derived by crossing a parent plant with a maternal haploid inducer.

5. The method of claim 4, wherein the advancing of at least one population in the breeding pipeline comprises creating a doubled-haploid plant from a haploid embryo that appeared in set 1.

6. The method of claim 5, wherein less than 20% of the populations of set 2 are selected for advancement in the breeding pipeline in step d.

7. The method of claim 5, wherein less than 30% of the populations of set 2 are selected for advancement in the breeding pipeline in step d.

8. The method of claim 5, wherein less than 40% of the populations of set 2 are selected for advancement in the breeding pipeline in step d.

9. The method of claim 5, wherein less than 50% of the populations of set 2 are selected for advancement in the breeding pipeline in step d.

10. The method of claim 5, wherein less than 60% of the populations of set 2 are selected for advancement in the breeding pipeline in step d.

11. The method of claim 5, wherein less than 70% of the populations of set 2 are selected for advancement in the breeding pipeline in step d.

12. The method of claim 5, wherein less than 80% of the populations of set 2 are selected for advancement in the breeding pipeline in step d.

13. The method of claim 5, wherein less than 90% of the populations of set 2 are selected for advancement in the breeding pipeline in step d.

14. The method of claim 5, wherein less than 95% of the populations of set 2 are selected for advancement in the breeding pipeline in step d.

15. The method of claim 1, wherein the step of determining which plants contain a desired trait is performed on plants after the plants have germinated.

16. The method of claim 1, wherein the step of determining which plants contain a desired trait comprises assessing the yield potential, disease resistance, or other performance data of a parent, ancestor, progeny, or other related germplasm growing in a field, greenhouse, or other area suitable for growing plants.

17. The method of claim 1, wherein performing step c. comprises genotyping to determine whether a plant contains a certain trait.

18. The method of claim 1, wherein the genotyping comprises using a molecular marker.

19. The method of claim 1, wherein the genotyping comprises detecting or quantifying the presence of a nucleic acid sequence produced by a plant in set 1.

20. The method of claim 1 wherein the rapid haploid sorter system comprises sorting seeds using data derived from performing an NMR analysis on at least one of the seeds from set 2.

21. The method of claim 20 wherein the rapid haploid sorter system detects an NMR signal associated with the expression of seed oil content.

22. The method of claim 1 wherein at least 10,000 seeds are haploid sorted in the breeding pipeline during any 12-month period.

23. A method of creating populations of doubled haploid plants in a doubled haploid production pipeline wherein a rapid haploid seed sorter is used to sort populations of haploid seeds derived from inducer crosses and wherein the number of haploid populations sorted is greater than the number of haploid populations that are advanced onto later steps of the breeding pipeline.

24. The method of claim 23, wherein advancing a population in the breeding pipeline comprises doubling the chromosome number of at least one cell of at least one embryo in the population.

25. The method of claim 24, wherein the number of induced haploid populations that are sorted in the breeding pipeline exceeds the number of induced haploid populations that are advanced in the pipeline by a factor of 10% during any 12-month period of time.

26. The method of claim 24, wherein the number of induced haploid populations that are sorted in the breeding pipeline exceeds the number of induced haploid populations that are advanced in the pipeline by a factor of 30% during any 12-month period of time.

27. The method of claim 24, wherein the number of induced haploid populations that are sorted in the breeding pipeline exceeds the number of induced haploid populations that are advanced in the pipeline by a factor of 50% during any 12-month period of time.

28. The method of claim 24, wherein the number of induced haploid populations that are sorted in the breeding pipeline exceeds the number of induced haploid populations that are advanced in the pipeline by a factor of 80% during any 12-month period of time.

29. The method of claim 24, wherein the number of induced haploid populations that are sorted in the breeding pipeline exceeds the number of induced haploid populations that are advanced in the pipeline by a factor of 100% during any 12-month period of time.

30. The method of claim 24, wherein the number of induced haploid populations that are sorted in the breeding pipeline exceeds the number of induced haploid populations that are advanced in the pipeline by a factor of 500% during any 12-month period of time.

31. A high-throughput method for bulking a population of doubled haploid seeds, the method comprising:
providing a first population of seeds comprising haploid seeds;
sorting the first population of seeds using a rapid haploid sorter system to accumulate a second population of seeds comprising haploid seeds, wherein the second population of seeds is substantially devoid of diploid seeds;
selecting one or more individual seeds exhibiting at least one preferred characteristic from the second population of seeds;
producing doubled haploid seeds from the selected seeds; and
cultivating plants or plant tissue from the selected doubled haploid seeds.
